(19) United States
(12) Patent Application Publication
(10) Pub. No.: US 2002/0177232 A1
(43) Pub. Date: Nov. 28, 2002

(54) Method and apparatus for detecting illicit substances

(76) Inventors: Richard J. Melker, Gainesville, FL (US); Bruce A. Goldberger, Gainesville, FL (US); Mark Gold, Gainesville, FL (US)

Correspondence Address: SALIWANCHIK LLOYD & SALIWANCHIK, A PROFESSIONAL ASSOCIATION, 2421 N.W. 41ST STREET, SUITE A-1, GAINESVILLE, FL, 326066669

(21) Appl. No.: 10/154,201

(22) Filed: May 22, 2002

Related U.S. Application Data
(60) Provisional application No. 60/292,962, filed on May 23, 2001.

(51) Int. Cl. G01N 33/00

(52) U.S. Cl. 436/151; 436/173; 422/83; 422/82.01

(57) ABSTRACT

The present invention includes a method and apparatus for detecting use of illicit substances by analyzing a sample of breath using electronic sensor technology, including surface acoustic-wave gas sensor technology. The method determines the presence and concentration of the substance (or a class of substances). Diagnostic software is used to identify substances where a stored library of signatures is compared to the signature obtained from the system. Signal processing and neural networks are preferably utilized in the analysis.

### CROSS-REFERENCE TO A RELATED APPLICATION

This application claims the benefit of U.S. Provisional Application Serial No. 60/292,962, filed May 23, 2001, incorporated herein by reference.

### FIELD OF INVENTION

The present invention relates to the detection of illicit substances, and, more particularly, to a method and apparatus for the detection of illicit substances in exhaled breath utilizing a rapidly responding device.

### BACKGROUND INFORMATION

The health risks related to illicit use of drugs are well documented. One drug of recent concern is gamma-hydroxy-butyrate (GHB), the use of which leads to risks of coma and death. GHB (also known as Georgia Home Boy, Grievous Bodily Harm, G, Liquid X, and others) is used outside the United States as an anesthetic agent and treatment for narcolepsy. [Kam, P. C., F. F. Yoong, (1998) “Gamma-hydroxybutyric acid: an emerging recreational drug,” Anaesthesia 53:1195-1198]. In the United States it has been sold in health food stores and on the Internet, as gamma-butyrolactone, (converted in the body to GHB), for use by body builders because of its anabolic properties. GHB produces euphoria, disinhibition, and memory disorders. GHB dissolves in water and is easily carried to parties and dances. GHB is often taken in addition to other drugs (e.g., benzodiazepines and alcohol) enhancing its potential effect and toxicity. Routine urine screening does not detect GHB.

The health risks related to GHB are well documented. GHB has been purported to be an effective anti-narcoleptic, anesthetic, anorectic, sedative, rapid eye movement (REM) sleep inducer, as well as agent for the treatment of ischemic conditions, alcohol and opiate withdrawal. [Graeme, K. A., (2000) “New drugs of abuse, “Emerg. Med. Clin. North Am. 18:625-636]. Users of GHB have compared it to other CNS depressants like marijuana, alcohol, and diazepam. GHB is a common drug of abuse, and its use is frequently reported in drug-facilitated sexual assault cases. [Bismuth, C. et al., (1997) “Chemical submission: GHB, benzodiazepines, and other knock out drops,” J. Toxicol. Clin. Toxicol. 35:595-598; and Slaughter, L. (2000) “Involvement of drugs in sexual assault,” J. Reprod. Med. 45:425-430].

The adverse effects of GHB span the entire range of severity. [Shannon, M., L. S. Quang, (2000) “Gamma-hydroxybutyrate, gamma-butyrolactone, and 1,4-butanediol: a case report and review of the literature,” Pediatr. Emerg. Care. 16:435-440]. Experimental data in humans demonstrate a rather narrow therapeutic index. [Ingels M. et al., (2000) “Coma and respiratory depression following the ingestion of GHB and its precursors: three cases,” J. Emerg. Med. 19:47-50]. Many of the effects are dose-dependent; smaller doses of 10 mg/kg are associated with amnesia and hypotonia, while larger doses of 50-70 mg/kg lead to anesthesia, respiratory depression, seizures, and coma. [Ropero-Miller J. D. and B. A. Goldberger (1998) “Recreational drugs: Current trends in the 90's,” Clin. Lab. Med. 18:727-746]. The adverse effects are highly variable among individuals, typically requiring experimentation to obtain an optimal dose. [O'Connell T. et al. (2000) Gamma-hydroxybutyrate (GHB): a newer drug of abuse,” Am. Fam. Physician 62:2478-2483.] This variability of effects, coupled with the variability inherent in the crude methods of illicit manufacturing, makes GHB a dangerous drug to consume. Furthermore, combining GHB with other CNS depressants lead to potential synergistic actions, resulting in increased toxicity.

Clinical treatment of GHB overdose includes supportive care and enhanced elimination. Spontaneous recovery occurs usually within 4-6 hours. In addition to causing acute effects, a withdrawal syndrome related to the chronic use of GHB is also of concern. [Dyer J. E. et al. (2001) “Gamma-hydroxybutyrate withdrawal syndrome,” Ann. Emerg. Med. 37:147-153].

Accordingly, there is an urgent need to develop a means to detect GHB in real-time, especially for use by emergency healthcare providers. GHB is not readily detected by the standard chemical tests utilized in hospital emergency departments or chemistry laboratories. Further, on-site test devices for GHB detection are not presently available. Reference laboratories using sophisticated techniques such as gas chromatography-mass spectrometry typically conduct complex toxicological analyses to determine the presence and quantity of GHB. While chemical analyses are complicated by endogenous GHB, the levels found immediately following overdose are usually comparably very high.

As such, there is a need for a real-time detector for GHB and other illicit drugs. Because emergency patients are often unconscious, there is a need for a detector that is capable of being used on patients in such a state. There is also a need for a GHB and other illicit drug sensor system capable of being used at remote locations to monitor the progress of recovering abusers.

All patents, patent applications, provisional applications, and publications referred to or cited herein, or from which a claim for benefit of priority has been made, are incorporated herein by reference in their entirety to the extent they are not inconsistent with the explicit teachings of this specification.

### BRIEF SUMMARY OF THE INVENTION

The present invention solves the problems in the art by providing a method and apparatus for detecting GHB and other illicit or controlled substances by providing a device for analyzing the patient's breath to confirm the presence of the substance. The substances detected by the present invention include, but are not limited to, illicit, illegal, and/or controlled substances, including drugs of abuse (amphetamines, analgesics, barbiturates, club drugs, cocaine, crack cocaine, depressants, designer drugs, ecstasy, Gamma Hydrixy Butyrate—GHB, Hallucinogens, Heroin/Morphine, Inhalants, Ketamine, Lysergic Acid Diethylamide—LSD, Marijuana, Methamphetamines, Opiates/Narcotics, Phencyclidine—PCP, Prescription Drugs, Psychedelics, Rohypnol, Steroids, and Stimulants). As used throughout the application and claims, reference to illicit substances is intended to include the above-noted broad description of substances.

The advantages of the invention are numerous. First and foremost, for healthcare personnel, the invention provides for a method by which emergency room personnel can readily determine if someone is suffering from an overdose or has taken drugs for which the sensor has been programmed to detect. A resulting advantage of the ability to rapidly detect an illicit drug through a simple and efficient system is the ability to timely treat overdoses. The subject technology for the present invention is inexpensive and potentially has broad medical application for detecting a wide range of compounds (both licit and illicit) in exhaled breath.

In operation, the analysis of the patient's breath includes comparing the substance sensed in the patient's breath with a predetermined signature profile of the substance. The predetermined signature profile is associated with a specific drug or a class of drugs. The method may further include the step of capturing the patient's breath in a vessel prior to analysis as well as dehumidifying the patient's breath prior to analysis in a manner well known in the art. Breath can be captured from the patient's mouth or nose. The data resulting from analysis of the patient's breath preferably includes substance concentration. In certain instances, such as during a drug treatment program, a baseline spectrum for the patient may be identified. In a further embodiment, the analysis further includes detecting exhalation of the patient's breath with a sensor.

In a preferred embodiment, the patient's breath is analyzed to confirm the presence of the substance by sensor technology selected from semiconductor gas sensor technology, conductive polymer gas sensor technology, surface acoustic wave gas sensor technology, aptamers (aptamer biosensors), and amplifying fluorescent polymer (AFP) sensors. The sensor technology produces a unique electronic fingerprint to characterize the substance such that the presence and concentration of the substance is determined.

The preferred device of the present invention includes (a) a sensor having a surface exposed to the patient's breath and/or airway and comprising a material selectively absorptive of a chemical substance or group of substances; and (b) an analyzer, coupled to the sensor, for producing an electrical signal indicative of the presence of the substance. The analyzer is further operative to determine the approximate concentration of the substance.

In one embodiment, the sensor is a surface acoustic wave device, such as that disclosed in pending U.S. application Ser. No. 09/708,789 entitled “Marker Detection Method and Apparatus to Monitor Drug Compliance” of which applicant is a co-inventor, the description of which is incorporated herein by reference. The sensor device disclosed in U.S. Pat. No. 5,945,069 may also be utilized. The device detects a target substance of an illicit nature in expired breath having the following components: (a) a surface-acoustic wave sensor capable of detecting the presence of the target substance in expired breath, wherein the sensor responds to the target substance by a shift in the resonant frequency; (b) an oscillator circuit having the sensor as an active feedback element; (c) a frequency counter in communication with the oscillator circuit to measure oscillation frequency which corresponds to resonant frequency of the sensor; and (d) a processor for comparing the oscillation frequency with a previously measured oscillation frequency of the target substance and determining presence and concentration of the target substance therefrom.

In an alternate embodiment, the device detects a target substance of an illicit nature in expired breath having the following components: (a) a sensor having an array of polymers capable of detecting the presence of the target substance in expired breath, wherein the sensor responds to the target substance by changing the resistance in each polymer resulting in a pattern change in the sensor array; (b) a processor for receiving the change in resistance, comparing the change in resistance with a previously measured change in resistance, and identifying the presence of the target substance from the pattern change and the concentration of the substance from the amplitude. The processor can include a neural network for comparing the change in resistance with a previously measured change in resistance to find a best match.

The invention also includes a method of determining the rate of washout of a target substance of an illicit nature in expired breath by (a) obtaining a sample of expired breath at a first interval; (b) analyzing the sample with sensor technology to determine the concentration of the substance; (c) obtaining at least one additional sample of expired breath at a later interval; (d) analyzing said additional sample with sensor technology to determine the concentration of said substance; and (e) comparing the concentration of the first sample with the concentration of additional samples to determine rate of washout of the target substance. The method alternatively includes the step of using sensor technology to measure metabolites of the substance in the step of determining the concentration of said substance. This includes measuring metabolites only and/or measuring metabolites and the substance itself.

The device may also include a means for receiving air exhaled by the patient. Preferably the device comprises sensor technology selected from semiconductor gas sensor technology, conductive polymer gas sensor technology, or surface acoustic wave gas sensor technology.

In alternate embodiments, the patient's breath is analyzed to confirm the presence of the substance by a spectrophotometer or a mass spectrometer.

The method further includes the step of recording data resulting from analysis of the patient's breath. The method further includes the step of transmitting data resulting from analysis of the patient's breath.

Accordingly, it is an object of the present invention to detect substances, such as illicit drugs, by methods including, but not limited to, sensor technology (e.g., silicon chip technology).

It is a further object of the present invention to provide a reporting system capable of tracking results and alerting healthcare personnel and health officials.

Further objects and advantages of the present invention will become apparent by reference to the following detailed description of the invention and appended drawings.

### BRIEF DESCRIPTION OF THE DRAWINGS

FIG. 1 is a view of a gas sensor chip in accordance with the present invention.

FIG. 2 is a view of a chemoselective polymer coated SAW sensor designed for the measurement of exhaled breath in accordance with the present invention.

FIG. 3A is a chromatogram for gamma butyrolactone from VaporLab™ with preconcentrator produced in accordance with the present invention.

FIG. 3B is a gamma butyrolactone GBL chart produced in accordance with the present invention.

FIG. 4 shows a gas sensor system in accordance with one embodiment of the invention.

FIG. 5 shows a gas sensor system in accordance with another embodiment of the invention.

### DETAILED DESCRIPTION OF THE INVENTION

The present invention provides a method and apparatus for detecting illicit substances. The substance is detected by devices including but not limited to electronic noses, spectrophotometers to detect the substance's IR, UV, or visible absorbance or fluorescence, or mass spectrometers to detect the substance's characteristic mass display.

Gas Sensor Technology

The preferred sensor technology is based on surface acoustic wave (SAW) sensors. These sensors oscillate at high frequencies and respond to perturbations proportional to the mass load of certain molecules. This occurs in the vapor phase on the sensor surface. The resulting frequency shift is detected and measured by a computer. Usually, an array of sensors (4-6) is used; each coated with a different chemoselective polymer that selectively binds and/or absorbs vapors of specific classes of molecules. The resulting array, or “signature,” identifies specific compounds. Sensitivity of the arrays is dependent upon the homogeneity and thickness of the polymer coating.

The invention preferably utilizes gas sensor technology, such as the commercial devices referred to as “artificial noses” or “electronic noses.” An “electronic or artificial nose” is an instrument, which comprises a sampling system, an array of chemical gas sensors with differing selectivity, and a computer with an appropriate pattern-classification algorithm, capable of qualitative and/or quantitative analysis of simple or complex gases, vapors, or odors. Electronic noses have been used mostly in the food, wine and perfume industry where their sensitivity makes it possible to distinguish between grapefruit oil and orange oil and identify spoilage in perishable foods before the odor is evident to the human nose. While there has been little medical-based research and application, recent examples demonstrate the power of this non-invasive technique. For example, electronic noses can determine the presence of bacterial infection in the lungs by analyzing the exhaled gases of patients for odors specific to particular bacteria. See Hanson C. W., H. A. Steinberger (September 1997) “The use of a novel electronic nose to diagnose the presence of intrapulmonary infection,” Anesthesiology 87(3A):Abstract A269. In addition, a genitourinary clinic utilized an electronic nose to screen for, and detect bacterial vaginosis. With the appropriate training the clinic achieved a 94% success rate. See Chandiok S. et al. (1997) “Screening for bacterial vaginosis: a novel application of artificial nose technology,” Journal of Clinical Pathology 50(9):790-791. Further, bacterial species can also be identified with the technology based on organism specific odors. See Parry A. D. et al. (1995) “Leg ulcer odor detection identifies beta-haemolytic streptococcal infection,” Journal of Wound Care 4:404-406.

Exhaled breath is used for a variety of medical tests and measurements. Probably the most recognized are detectors for ethyl alcohol. Real-time measurement of end-tidal carbon dioxide concentration (etCO2), has proven to be a valuable tool for estimating arterial CO2 concentration. It is routinely used during anesthesia to replace invasive arterial or venous blood gas measurement. The technique is also used to detect exhaled anesthetic gas and oxygen concentration.

As previously stated, exhaled gas measurements can be used diagnostically. A breath test for ammonia can alert clinicians to the presence of Helicobacter pylori, as well as bacterial overgrowth of the small bowel and stomach. See Perri F. (2000) Diagnosis of Helicobacter pylori infection: which is the best test? The urea breath test, Dig. Liver. Dis. 32(Suppl 3):S196-198; and Ganga-Zandzou P. S. et al. (2001) A 13C-urea breath test in children with Helicobacter pylori infection: validity of the use of a mask to collect exhaled breath samples,” Acta. Paediatr. 90:232-233. Most breath tests are expensive, time consuming and must be performed under laboratory conditions by trained technicians.

A recent Defense Advanced Research Projects Agency (DARPA) initiative to improve landmine detection breakdown products resulted in several technologies designed to mimic the olfactory system (artificial nose) (http://www.darpa.mil/ato/programs/uxo/index.html). At present, dogs are generally used for landmine detection because of their ability to locate extremely low concentrations of the breakdown products of explosives. This gives rise to the project name, i.e.—the dog's nose project. These technologies operate by sensing vapors of breakdown products that are released into the soil and air. Among the competing technologies were ones capable of detecting breakdown products in the range of parts per trillion.

One technology for detection is based on the ability of volatile compounds to cause perturbations in the oscillation of surface acoustic wave (SAW) sensors. See Wohltjen, H., D. S. Ballantine, “Surface Acoustic Wave Devices for Chemical Analysis,” Analytical Chemistry 61:704A; and Fang, M.; K. Vetelino, M. Rothery, J. Hines, G. Frye, (1999) “Detection of Organic Chemicals by SAW Sensor Array,” Sensors and Actuators B56:155-157. A high degree of sensitivity and specificity can be achieved by coating the surface of the sensors with “chemoselective” polymers that react in a predictable manner with the target compound. See Wohltjen, H., D. S. Ballantine, N. L. Jarvis, (1989) “Vapor Detection with Surface Acoustic Wave Microsensors,” Chemical Sensors and Microinstrumentation, American Chemical Society, pp. 157-175; and Wohltjen, H.; D. S. Ballantine, A. Snow, J. W. Grate, M. H. Abraham, A. McGill, P. Sasson, “Determination of Partition Coefficients from Surface Acoustic Wave Vapor Sensor Responses and Correlation with Gas-Liquid Chromatographic Partition Coefficients,” Analytical Chemistry, 60(9):869-875. By using an adequate number of sensors and the appropriate polymers, unique “signatures” can be reproducibly detected for specific compounds in qualitative and quantitative measurements.

DARPA tests showed that one version of this technology was able to reliably recognize DNT (a breakdown product of TNT) at levels of 3.5 ppbv in dry air and between 10-15 ppbv in moisture saturated air (as is the case for exhaled breath). The range of applicability of this technology to chemical detection is limited only by the ability to develop, discover or design coatings for the SAW device that make it sensitive and selective for the analyte or target compound to be measured. When the appropriate coating is available, it is possible to detect vapors at the 10-100 ppbv concentration level within a few minutes with selectivity of 1000:1 or more over some commonly encountered interferences. A dynamic range of 3-4 orders of magnitude is common.

A number of patents which describe gas sensor technology include the following: U.S. Pat. No. 5,945,069 to Buchler, entitled “Gas sensor test chip”; U.S. Pat. No. 5,918,257 to Mifsud et al., entitled “Method and devices for the detection of odorous substances and applications”; U.S. Pat. No. 4,938,928 to Koda et al., entitled “Gas sensor”; U.S. Pat. No. 4,992,244 to Grate, entitled “Films of dithiolene complexes in gas-detecting microsensors”; U.S. Pat. No. 5,034,192 to Wrighton et al., entitled “Molecule-based microelectronic devices”; U.S. Pat. No. 5,071,770 to Kolesar, Jr., entitled “Method for gaseous component identification with #3 polymeric film”; U.S. Pat. No. 5,145,645 to Zakin et al., entitled “Conductive polymer selective species sensor”; U.S. Pat. No. 5,252,292 to Hirata et al., entitled “Ammonia sensor”; U.S. Pat. No. 5,605,612 to Park et al., entitled “Gas sensor and manufacturing method of the same”; U.S. Pat. No. 5,756,879 to Yamagishi et al., entitled “Volatile organic compound sensors”; U.S. Pat. No. 5,783,154 to Althainz et al., entitled “Sensor for reducing or oxidizing gases”; and U.S. Pat. No. 5,830,412 to Kimura et al., entitled “Sensor device, and disaster prevention system and electronic equipment each having sensor device incorporated therein,” all of which are incorporated herein by reference in their entirety.

Recent developments in the field of detection non-exclusively include: semiconductive gas sensors; mass spectrometers, and IR, UV, visible, or fluorescence spectrophotometers. The substances change the electrical properties of the semiconductors by making their electrical resistance vary, and the measurement of these alternatives allows one to determine the concentration of substances. These methods and apparatus used for detecting substances have brief detection time of a few seconds. This short detection time is more desirable compared to those given by gas chromatography, which takes from several minutes to several hours.

Other recent gas sensor technologies included in the present invention include apparatus having conductive-polymer gas-sensors (“polymeric”), apparatus having surface-acoustic-wave (SAW) gas-sensors, and aptamers (aptamer biosensors), and amplifying fluorescent polymer (AFP) sensors.

The conductive-polymer gas-sensors (also referred to as “chemoresistors”) are coated with a film sensitive to the molecules of certain odorous substances. On contact with the molecules, the electric resistance of the sensors change and the measurement of the variation of this resistance enables the concentration of the target substances to be determined. An advantage of this type of sensor is that it functions at temperatures close to ambient. One can also obtain different sensitivities for detecting different odorous substances by modifying or choosing an alternate conductive polymer.

Polymeric gas sensors can be built into an array of sensors, where each sensor responds to different gases and augment the selectivity of the odorous substances.

The surface-acoustic-wave (SAW) gas-sensors generally include a substrate with piezoelectric characteristics covered by a polymer coating, which is able to selectively absorb the target substances. The variation of the resulting mass leads to a variation of its resonant frequency. This type of sensor provides very good mass-volume measures of the odorous substances. In the SAW device, the substrate is used to propagate a surface acoustic wave between sets of interdigitated electrodes. The chemoselective material is coated on the surface of the transducer. When a chemical analyte interacts with the chemoselective material coated on the substrate, the interaction results in a change in the SAW properties, such as the amplitude or velocity of the propagated wave. The detectable change in the characteristics of the wave indicates the presence and concentration of the chemical analyte.

SAW devices are described in numerous patents and publications, including U.S. Pat. No. 4,312,228 to Wohltjen; U.S. Pat. No. 4,895,017 to Pyke and Groves W A, et al. (1988) “Analyzing organic vapors in exhaled breath using surface acoustic wave sensor array with preconcentration: Selection and characterization of the preconcentrator adsorbent,” Analytica Chimica Acta 371:131-143, all of which are incorporated herein by reference. Other types of chemical sensors known in the art that use chemoselective coatings applicable to the operation of the present invention include bulk acoustic wave (BAW) devices, plate acoustic wave devices, interdigitated microelectrode (IME) devices, optical waveguide (OW) devices, electrochemical sensors, and electrically conducting sensors.

The operating performance of a chemical sensor that uses a chemoselective film coating is greatly affected by the physical characteristics of the coating. Thickness, uniformity and composition are all factors that effect testing accuracy. For some biosensors, increase or fluctuations in the coating thickness, can have a detrimental effect on the sensitivity. This occurs because the portion of the coating immediately adjacent to the transducer substrate is sensed by the transducer. If the polymer coating is too thick, the sensitivity of the SAW device to record changes in frequency is reduced. This is caused by the outer layers of coating material competing for the analyte with the layers of coating.

Uniformity of the chemoselective coating is also a critical factor in the performance of a sensor. Changes in surface area can greatly affect the local vibrational signature of the SAW device. Therefore, films should be deposited that are consistent to within 1 nm with a thickness of 15-25 nm. In this regard, it is important that the coating be uniform and reproducible from one device to another, but also that the coating on a single device be uniform across the active area of the substrate. This ensures that a set of devices will all operate with the same sensitivity. If a coating is non-uniform, the response time to analyte exposure and the recovery time after analyte exposure are increased and the operating performance of the sensor is impaired. The thin areas of the coating respond more rapidly to an analyte than the thick areas. As a result, the sensor response signal takes longer to reach an equilibrium value, and the results are less accurate than they would be with a uniform coating.

Most current technologies for creating large area films of polymers and biomaterials involve spinning, spraying, or dipping a substrate into a solution of the macromolecule and a volatile solvent. These methods coat the entire substrate without selectivity and sometimes lead to solvent contamination and morphological inhomogeneities in the film due to non-uniform solvent evaporation. There are also techniques such as microcontact printing and hydrogel stamping that enable small areas of biomolecular and polymer monolayers to be patterned, but separate techniques like photolithography or chemical vapor deposition are needed to transform these films into microdevices. Other techniques such as thermal evaporation and pulsed laser ablation are limited to polymers that are stable and not denatured by vigorous thermal processes. More precise and accurate control over the thickness and uniformity of a film coating may be achieved by using pulsed laser deposition (PLD), a physical vapor deposition technique that has been developed recently for forming ceramic coatings on substrates. By this method, a target comprising the stoichiometric chemical composition of the material to be used for the coating is ablated by means of a pulsed laser, forming a plume of ablated material that becomes deposited on the substrate.

Polymer thin films, using a new laser based technique developed by researchers at the Naval Research Laboratory called Matrix Assisted Pulsed Laser Evaporation (MAPLE), have recently been shown to increase sensitivity and specificity of chemoselective SAW vapor sensors. A variation of this technique, Pulsed Laser Assisted Surface Functionalization (PLASF) is preferably used to design compound specific biosensor coatings with increased sensitivity for the present invention. PLASF produces similar thin films for sensor applications with bound receptors or antibodies for biosensor applications. This provides improved SAW biosensor response by eliminating film imperfections induced by solvent evaporation and detecting molecular attachments to specific antibodies. This results in high sensitivity and specificity.

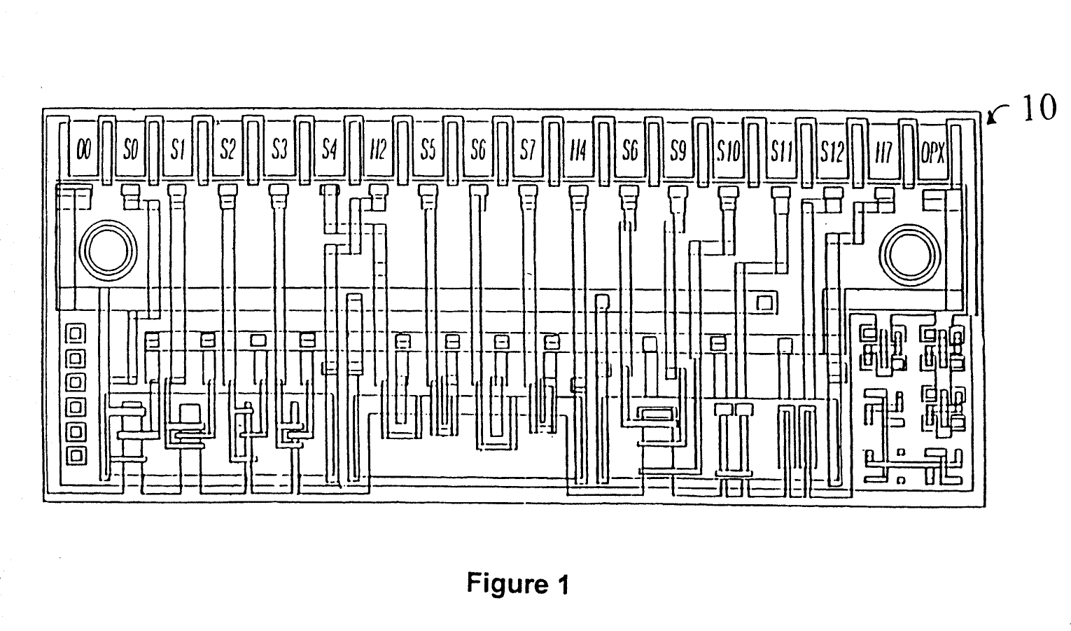

Certain extremely sensitive, commercial off-the-shelf (COTS) electronic noses, such as those provided by Cyrano Sciences, Inc. (ACSI”) (e.g., CSI's Portable Electronic Nose and CSI's Nose-Chip™ integrated circuit for odor-sensing—U.S. Pat. No. 5,945,069—FIG. 1), are preferred in the present invention to monitor the exhaled breath from a patient. These devices offer minimal cycle time, can detect multiple odors, can work in almost any environment without special sample preparation or isolation conditions, and do not require advanced sensor design or cleansing between tests.

Other technologies and methods are contemplated herein for detection of substances. For example, a patient's breath can be captured into a container (vessel) for later analysis at a central instrument such as a mass spectrometer.

Aptamers (aptamer biosensors) may be utilized in the present invention for sensing. Aptamer biosensors are resonant oscillating quartz sensors which can detect minute changes in resonance frequence due to modulations of mass of the oscillating system which results from a binding or dissociation event.

Similarly, amplifying fluorescent polymer (AFP) sensors may be utilized in the present invention for sensing. AFP sensors are an extremely sensitive and highly selective chemosensors that use amplifying fluorescent polymers (AFPs). When vapors bind to thin films of the polymers, the fluorescence of the films decreases. A single molecular binding event quenches the fluorescence of many polymer repeat units, resulting in an amplification of the quenching. Analyte binding to the films is reversible, so the films can be reused.

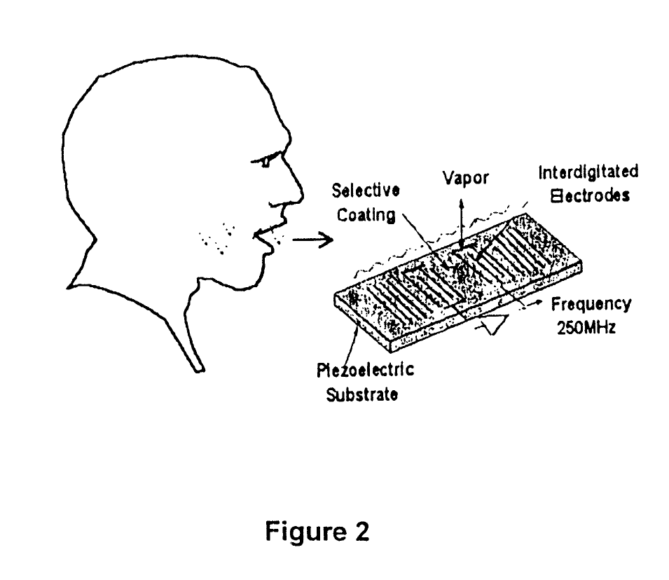

FIG. 2 is an illustration of a chemoselective polymer coated SAW sensor designed for the measurement of exhaled breath vapor.

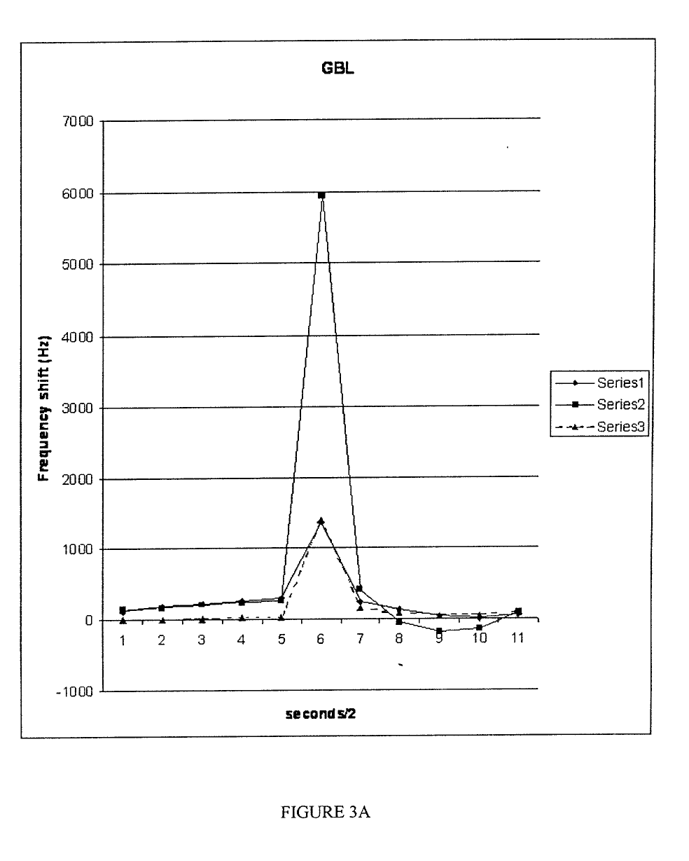

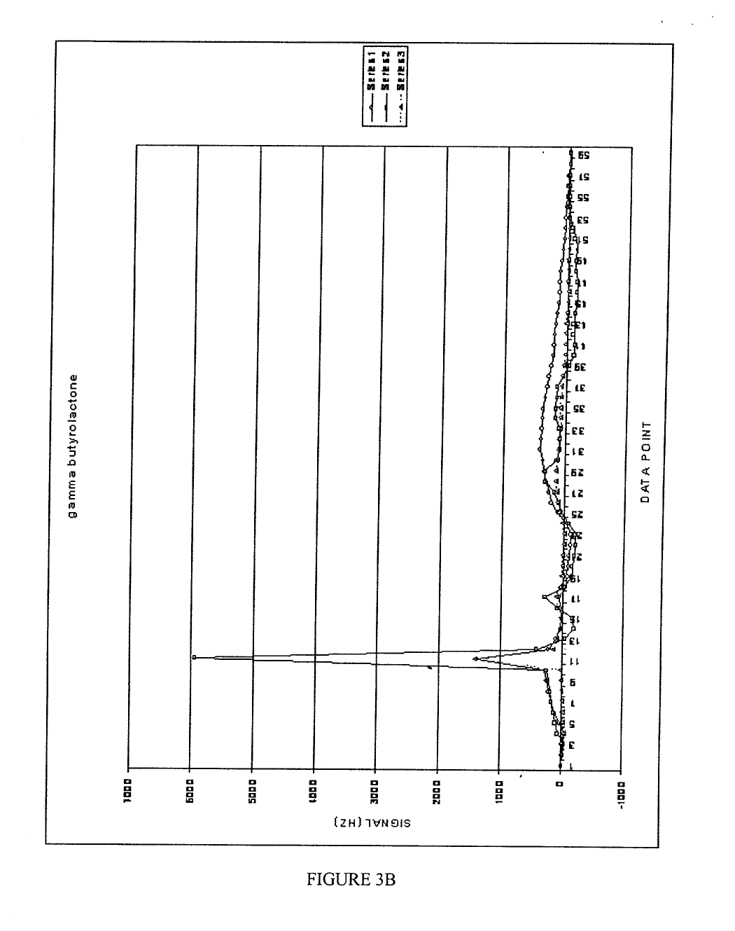

FIGS. 3A-3B show a chromatogram for gamma butyrolactone from VaporLab™ with preconcentrator produced in accordance with the present invention and a gamma butyrolactone GBL chart, respectively. Note that the “signature” has both amplitude and temporal resolution. In the present invention, vapor concentration measurements of vapors (analytes) are made by detecting the adsorption of molecules onto the surface of a SAW sensor coated with a polymer thin film. This thin film is specifically coated to provide selectivity and sensitivity to specific analytes. The SAW is inserted as an active feedback element in an oscillator circuit. A frequency counter measures the oscillation frequency, which corresponds to the resonant frequency of the SAW sensor. The response of the SAW sensor to the analyte is measured as a shift in the resonant frequency of the SAW sensor. This configuration requires an oscillator circuit, the coated SAW sensor, and a frequency counter, all of which can be housed on a small printed circuit board.

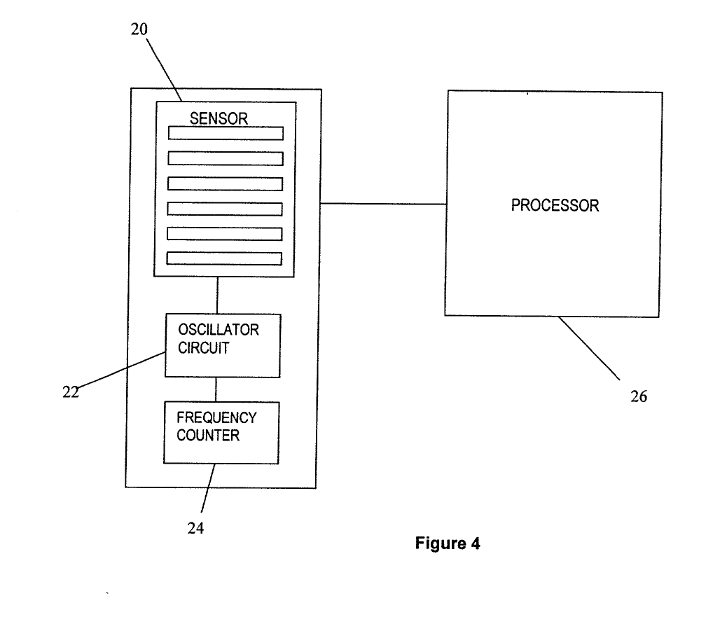

FIG. 4 shows an example of a device for detecting a target substance of an illicit nature in expired breath having the following components: (a) a surface-acoustic wave sensor 20 capable of detecting the presence of the target substance in expired breath, wherein the sensor responds to the target substance by a shift in the resonant frequency; (b) an oscillator circuit 22 having the sensor as an active feedback element; (c) a frequency counter 24 in communication with the oscillator circuit to measure oscillation frequency which corresponds to resonant frequency of the sensor; and (d) a processor 26 for comparing the oscillation frequency with a previously measured oscillation frequency of the target substance and determining presence and concentration of the target substance therefrom. The sensor can include measuring circuitry (not shown) and an output device (not shown) can also be included (e.g., screen display, audible output, printer).

The processor can include a neural network (not shown) for pattern recognition. Artificial Neural Networks ANNs are self learning; the more data presented, the more discriminating the instrument becomes. By running many standard samples and storing results in computer memory, the application of ANN enables the device to “understand” the significance of the sensor array outputs better and to use this information for future analysis. “Learning” is achieved by varying the emphasis, or weight, that is placed on the output of one sensor versus another. The learning process is based on the mathematical, or “Euclidean,” distance between data sets. Large Euclidean distances represent significant differences in sample-to-sample aroma characteristics.

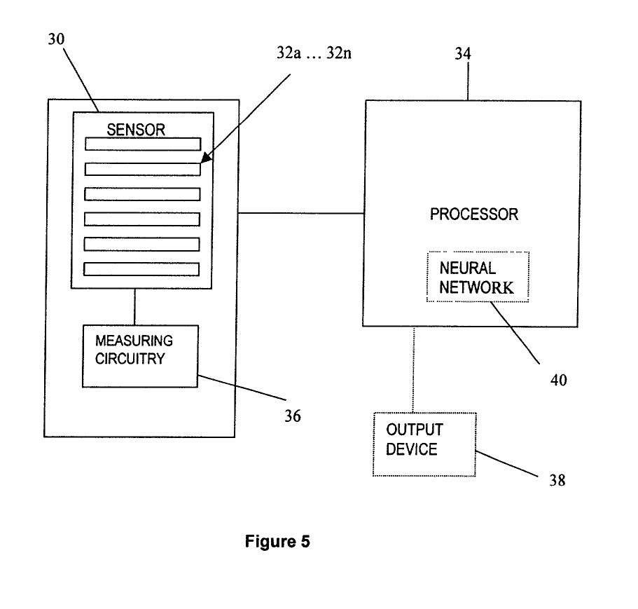

In an alternate embodiment, FIG. 5 shows an example of a device for detecting a target substance of an illicit nature in expired breath having the following components: (a) a sensor 30 having an array of polymers 32a-32n capable of detecting the presence of the target substance in expired breath, wherein the sensor responds to the target substance by changing the resistance in each polymer resulting in a pattern change in the sensor array; (b) a processor 34 for receiving the change in resistance, comparing the change in resistance with a previously measured change in resistance, and identifying the presence of the target substance from the pattern change and the concentration of the substance from the amplitude. The processor can include a neural network 40 for comparing the change in resistance with a previously measured change in resistance to find a best match (pattern recognition). The sensor can include measuring circuitry 36 and an output device 38 can also be included (e.g., screen display, audible output, printer).

The present invention will determine if a person has ingested any substance by monitoring and analyzing the exhaled gases with the electronic nose and comparing these measurements against a library of chemical substances and interferents. In a preferred embodiment, the device of the present invention is designed so that patients can exhale via the mouth or nose directly into the device.

Another preferred electronic nose technology of the present invention comprises an array of polymers, for example, 32 different polymers, each exposed to a substance. Each of the 32 individual polymers swells differently to the substance creating a change in the resistance of that membrane and generating an analog voltage in response to that specific substance (“signature”). Based on the pattern change in the sensor array, the normalized change in resistance is then transmitted to a processor to identify the type, quantity, and quality of the substance. The unique response results in a distinct electrical fingerprint characterizing the substance. The pattern of resistance changes of the array indicates the presence of the target substance and the amplitude of the pattern indicates its concentration.

This technology can be used to identify classes of illicit substances (e.g., amphetamines, barbiturates, canniboids, benzodiapines, opiates, etc . . . ) by determining first the signature for each class of substances as well as specific substances. In the case of the GHB detector, a signature for GHB is first determined. In addition, a library of interferent signatures is created to allow the sensor to discriminate the GHB signal from background noise.

The responses of the electronic nose to specific substances are fully characterized using a combination of conventional gas sensor characterization techniques. For example, the sensor can be attached to a computer where marker analysis results are displayed on the computer screen, stored, transmitted, etc. A data analyzer compares the pattern of response to previously measured responses from known substances. The pattern matching can be performed using a number of techniques, including neural networks. By comparing the analog output from each of the 32 polymers to a “blank” or control substance, a neural network can establish a pattern, which is unique to that substance and subsequently learns to recognize that substance. The particular resistor geometries are selected to optimize the desired response to the particular substance being sensed. The electronic nose of the present invention is preferably a self-calibrating polymer system suitable for liquid or gas phase biological solutions for a variety of substances simultaneously.

The electronic nose of the present invention can include integrated circuits (chips) manufactured in a modified vacuum chamber for Pulsed Laser Deposition of polymer coatings. It can operate the simultaneous thin-film deposition wave detection and obtain optimum conditions for high sensitivity of SAW sensors. The morphology and microstructure of biosensor coatings is characterized as a function of process parameters.

The electronic nose used in the present invention is preferably designed so that patients can exhale directly into the device. For example, a mouthpiece or nosepiece will be provided for interfacing a patient with the device to readily transmit the exhaled breath to the sensor (See, e.g., U.S. Pat. No. 5,042,501). This, however, is not a limitation on the invention as breath samples can be both, sampled immediately or stored. The output from the neural network of the modified electronic nose is similar when the same patient exhales directly into the device and when the exhaled gases are allowed to dry, before they are sampled by the electronic nose.

The humidity in the exhaled gases represents a problem for certain electronic nose devices (not, however, SAW sensors) because they will only work with “dry” gases. When using such humidity sensitive devices, the present invention includes a means to dehumidify the samples. This is accomplished by including a commercial dehumidifier or a heat moisture exchanger (HME), a device designed to prevent desiccation of the airway during ventilation with dry gases. Alternatively, the patient may exhale through their nose, which is an anatomical, physiological dehumidifier to prevent dehydration during normal respiration.

However, there may be instances where detection after excretion from the lungs is preferable. This may be the case when a substance is taken by the intravenous route. Under these circumstances, excretion may occur rapidly since intravenously injected substances pass rapidly to the lungs.

Thus, when a substance is ingested, the preferred embodiment of the invention detects the presence of that substance almost immediately in the exhaled breath of the person (or possibly by requesting the person to deliberately produce a burp) using the electronic nose. The electronic nose can determine the presence of a substance as well as its concentration. Therefore, electronic noses can not only detect if substances are there, but also how much of the substance is there.

Preferably, operating in conjunction with a drug monitoring program, the electronic nose is used to identify a baseline spectrum for the patient without illicit drugs in his system, if necessary. This will prove beneficial for the detection of more than one substance if the patient ingests more than one drug at a time as well as possible interference from different foods and odors in the stomach, mouth, esophagus and lungs.

When the drugs are ingested, they are dissolved in the mouth (or digested in the stomach, transmitted to the lungs, etc.). The electronic nose then detects the drug when the patient exhales. The electronic nose can record and/or transmit the data sensed from the patient's breath for monitoring purposes.

A pressure sensor can also be incorporated into the detector to confirm that the patient is actually exhaling into the device. A flow restrictor can be incorporated thereby increasing the resistance to exhalation. Adding a pressure transducer to the system, a pressure change from baseline can be measured during exhalation. Furthermore, a number of detectors are available (i.e. end-tidal carbon dioxide monitors) that can be added to the device.

The electronic nose and/or computer communicating therewith can also notify the medical staff and/or the patient to any irregularities in dosing, dangerous drug interactions, and the like. Furthermore, this system will confirm whether a patient has taken a specific substance.

Remote Communication System

A further embodiment of the invention includes a communications device in the home (or other remote location) that is interfaced to the electronic nose. This device can be used to monitor subject compliance with treatment regimens or abstinence. The home communications device can transmit the data collected by the compliance-monitoring device immediately or at prescribed intervals directly or over a standard telephone line (or other communication means). The communication of the data will allow the physician to be able to remotely verify the results. The data transmitted from the home can also be downloaded to a computer and stored in a database, and any problems would be automatically flagged (e.g., alarm). Such a system may include additional features as described in the system disclosed in U.S. Pat. No. 6,074,345, incorporated herein by reference.

Following are examples which illustrate procedures for practicing the invention. These examples should not be construed as limiting.

### EXAMPLE 1

### Sensor Design for GHB Testing

Pure solutions of GHB are acquired and tested on a Microsensor Systems VaporLab™ detector that has been optimized for GHB. [Microsensor Systems, Inc., of Bowling Green, Ky., USA makes commercial SAW based detectors for industry and the military.] The existing sensor system is modified to include an array of polymer coatings for the optimal combination of polymers and sensor numbers to provide the best available “signature”. Samples of GHB are diluted using a “copper kettle” vaporizer and calibrated gas flow meters, using air as the diluting gas. Calibration curves for GHB are then determined, thus creating a signature for the suspect compound. Thereafter SAW sensors are used to determine the presence and/or concentration of the suspect compound or analyte in a gas sample.

In order to ensure the accuracy and integrity of the sensor measurements, the sensor is calibrated both qualitatively and quantitatively with an accepted protocol. A headspace autosampler for gas chromatography (GC), in conjunction with a gas mixer, is used to correlate the GC and sensor array responses to different concentrations of the gas samples. Samples are diluted with an aqueous solution containing an appropriate internal standard and placed into a sealed vial suitable for headspace analysis. The samples along with appropriate standards are incubated at an elevated temperature allowing volatiles to diffuse out of the liquid layer (sample phase) as vapors into the “headspace” (gas phase) within the sealed vial. Under constant conditions of temperature, pressure and equilibration time, the vapor phase in each of these vials is sequentially sampled and separated on a suitable gas chromatographic capillary column. The volatile components are detected using a flame ionization detector or nitrogen phosphorous detector. A library of interferents is created by mixing samples of the interferents found in exhaled breath and analyzing the samples with and without the addition of GHB. This example, however, is not limited to GHB as any other illicit substance can be tested using this method by substituting that specific substance for GHB.

### EXAMPLE 2

### Diagnostic Software Development

Diagnostic software can identify compounds, and in the case of the detection of GHB, a library of signatures is recorded to compare against the signatures obtained from the sensor system. The software includes complex signal processing/neural networks. The system distinguishes GHB from interferents normally found in exhaled breath. Once the signature of GHB is known, samples of exhaled breath are taken at various times during the day and on multiple days. The samples are analyzed for interferents, known concentrations of analytes are added to exhaled breath samples to calibrate the system to detect GHB in the presence of interferents.

Multiple sensors address the broad response of the sensing technology and guarantee selectivity (statistical detection). Statistical pattern recognition divides the full measurement space into a set of regions that are assigned to each class. However, detection theory recognizes that only part of the measurement space is known, and proposes methods to discriminate among known classes and further between the known classes and the background.

In order to address the sensing of chemicals from the environment, a two stage processing system is used: First a segmentation stage (where the system essentially asks, “is there a new chemical?”) followed by a pattern recognition stage (where the system essentially asks, “given that there is a new chemical, which is it?”). This is the way statistical detection theory suggests dealing with uncertainty.

Similar concepts are used for chemical sensing. One difference is that in chemical sensing there will be a time series instead of an image. To clarify, the local CFAR (Constant False Alarm Rate) properties are translated in the statistical local variations of the time series, which are measured by what is referred to as the Generalized Likelihood Ratio Test (GLRT). The GLRT is extended with neural networks to produce a fine segmentation algorithm called competitive mixture of experts. This is the methodology applied to chemical sensing. Basically, the system will segment the incoming signal in regions that change statistically from the previous ones. Thus, if the chemical composition in the air does not change the system it will be “called” the background activity. Once there is a statistical change from the previous segment, then the algorithm will segment the nonstationary portion of the time series and present it to a classifier that will identify it as one of the substances (or unknown). This second stage is also based on a neural network classifier. There are several to choose from. A neural topology, which implements local decision regions in pattern space, is preferable to global discriminants. The new support vector machine (SVM) classifier is preferably applied. As an alternative, a methodology developed in the University of Florida Computational NeuroEngineering Laboratory (CNEL) that finds information relevant features from the data before classification may be used. This method has been also been shown to be very sensitive and specific in real world classification problems. Once the optimal polymers are determined, thin, homogeneously coated SAW sensors are produced using PLASF. This improved polymer deposition technique should optimize the SAW responses to the analytes. The detector preferably can distinguish a single or multiple analytes from a background of interferents. Samples of exhaled breath are collected in non-porous vessels (likely glass) and onto silica gel (in glass traps) at specific intervals following drug administration. The intervals are from the time of the last GHB dose in order to evaluate the time course of the washout of GHB. The preserved samples are analyzed as described above. The rate of disappearance of GHB from the breath will be temporally analyzed.

Inasmuch as the preceding disclosure presents the best mode devised by the inventor for practicing the invention and is intended to enable one skilled in the pertinent art to carry it out, it is apparent that methods incorporating modifications and variations will be obvious to those skilled in the art. The substances detected by the present invention include, but are not limited to, illicit, illegal, and/or controlled substances, including drugs of abuse (amphetamines, analgesics, barbiturates, club drugs, cocaine, crack cocaine, depressants, designer drugs, ecstasy, Gamma Hydrixy Butyrate—GHB, Hallucinogens, Heroin/Morphine, Inhalants, Ketamine, Lysergic Acid Diethylamide—LSD, Marijuana, Methamphetamines, Opiates/Narcotics, Phencyclidine—PCP, Prescription Drugs, Psychedelics, Rohypnol, Steroids, and Stimulants). As used throughout the application and claims, reference to illicit substances is intended to include the above-noted broad description of substances. As such, it should not be construed to be limited thereby but should include such aforementioned obvious variations and be limited only by the spirit and scope of the following claims.

References

1. Graeme, K. A. (2000) “New drugs of abuse,” Emerg. Med. Clin. North Am. 18:625-636.

2. Dyer, J. E. (1991) Ag-Hydroxybutyrate: a health-food product producing coma and seizurelike activity,” Am. J. Emerg. Med. 9:321-324.

3. McCusker, R., H. Paget-Wilkes, C. Chronister, B. A. Goldberger, and M. A. ElSohly (1999) “Analysis of gamma-hydroxybutyrate (GHB) in urine by gas chromatography/mass spectrometry,” J. Analyt. Toxicol. 23:301-305.

4. Kam, P. C., F. F. Yoong (1998) “Gamma-hydroxybutyric acid: an emerging recreational drug,” Anaesthesia 53:1195-1198. Review

5. Bismuth, C., S. Dally, S. W. Borron (1997) “Chemical submission: GHB, benzodiazepines, and other knockout drops,” J. Toxicol. Clin. Toxicol. 35:595-598.

6. Slaughter, L. (2000) “Involvement of drugs in sexual assault,” J. Reprod. Med. 45:425-430.

7. Shannon, M., L. S. Quang (2000) “Gamma-hydroxybutyrate, gamma-butyrolactone, and 1,4-butanediol: a case report and review of the literature,” Pediatr. Emerg. Care. 16:435-440. Review.

8. Ingels, M., C. Rangan, J. Bellezzo, R. F. Clark (2000) “Coma and respiratory depression following the ingestion of GHB and its precursors: three cases,” J. Emerg. Med. 19:47-50.

9. Ropero-Miller, J. D. and B. A. Goldberger (1998) “Recreational drugs: Current trends in the 90's,” Clin. Lab. Med. 18:727-746.

10. O'Connell, T., L. Kaye, J. J. Plosay (2000) “Gamma-hydroxybutyrate (GHB): a newer drug of abuse,” Am. Fam. Physician. 62:2478-2483. Review.

11. Dyer, J. E., B. Roth, B. A. Hyma (2001) “Gamma-hydroxybutyrate withdrawal syndrome,” Ann. Emerg. Med. 37:147-153.

12. Perri, F. (2000) “Diagnosis of Helicobacter pylori infection: which is the best test? The urea breath test,” Dig. Liver Dis. 32(Suppl 3):S196-198.

13. Ganga-Zandzou, P. S., P. Vincent, L. Michaud, D. Guimber, D. Turck, F. Gottrand (2001) “13C-urea breath test in children with Helicobacter pylori infection: validity of the use of a mask to collect exhaled breath samples,” Acta. Paediatr. 90:232-233.

14. Wohltjen, H., D. S. Ballantine, “Surface Acoustic Wave Devices for Chemical Analysis,” Analytical Chemistry 61:704A.

15. Fang, M., K. Vetelino, M. Rothery, J. Hines, G. Frye (1999) “Detection of Organic Chemicals by SAW Sensor Array,” Sensors and Actuators B56:155-157.

16. Wohltjen, H., D. S. Ballantine, N. L. Jarvis (1989) “Vapor Detection with Surface Acoustic Wave Microsensors,” Chemical Sensors and Microinstrumentation; American Chemical Society, pp. 157-175.

17. Wohltjen, H., D. S. Ballantine, A. Snow, J. W. Grate, M. H. Abraham, A. McGill, P. Sasson, “Determination of Partition Coefficients from Surface Acoustic Wave Vapor Sensor Responses and Correlation with Gas-Liquid Chromatographic Partition Coefficients,” Analytical Chemistry 60(9):869-875.

18. Wohltjen, H., “Chemical Sensing with Surface Acoustic Wave (SAW) Devices,” Microsensor Systems, Inc. (http://www.microsensorsystems.com/pdf/chemicalsensing.pdf).

19. Bond, W. S. and D. A. Hussar (1991) “Detection methods and strategies for improving medication compliance,” Am. J. Hosp. Pharm. 48:1978-1988.

20. Cramer, J. A. (1995) “Optimizing long-term patient compliance,” Neurology 45(1):S25-S28.

21. Burman, W. J., D. L. Cohn, C. A. Reitmeijer, et al. (1997) “Noncompliance with directly observed therapy for tuberculosis,” Chest 111:1168-1173.

22. Burman, W. J., C. B. Dalton, D. L. Cohn, J. R. Butler, R. R. Reves (1997) “A cost-effectiveness analysis of directly observed therapy vs self-administered therapy for treatment of tuberculosis,” Chest 112:63-70.

23. Principe, J., M. Kim, J. Fisher (1998) “Target detection in synthetic aperture radar (SAR) using artificial neural networks,” IEEE Trans. Image Proc. special issue on neural networks, 7:1136-1149.

24. Principe, J., A. Radisavljevic, J. Fisher, M. Haytt, L. Novak (1999) “Target prescreening based on a quadratic gamma detector,” IEEE Trans. Aerospace. 34:706-715.

25. Haykin, S., I. Sandberg, E. Wan, J. Principe, C. Fancourt, S. Katagiri (2001) Nonlinear dynamical systems: Feedforward neural network perspective, John Wiley.

26. Principe, J., D. Xu, J. Fisher (2000) Information Theoretic Learning, in Unsupervised Adaptive Filtering, Simon Haykin Editor, pp. 265-319, Wiley.

27. Principe, J., N. Euliano, C. Lefebvre (2000) Neural Systems: Fundamentals through Simulations, CD-ROM textbook, John Wiley.

28. Zhao, Q., J. Principe, V. Brennan, D. Xu, Z. Wang (2000) “Synthetic aperture radar automatic target recognition with three strategies of learning and representation,” Optical Engineering 39(5):1230-1244.

29. Vapnik, V. (1998) The Nature of Statistical Learning Theory, Springer Verlag.

30. Zhao, Q., J. Principe, “Forming large margins with support vector machines for synthetic aperture radar automatic target recognition,” IEEE Trans. Aerospace and Elect Systems.

We claim:

1. A method of detecting illicit substances, comprising:
obtaining a sample of exhaled breath; and
analyzing said sample of breath with sensor technology to determine the presence of said illicit substance.

2. The method of claim 1 wherein said sample is analyzed to determine the presence of said illicit substance by sensor technology selected from the group consisting of: semiconductor gas sensor technology; conductive polymer gas sensor technology; aptamer sensor technology; amplifying fluorescent polymer (AFP) sensor technology; or surface acoustic wave gas sensor technology.

3. The method of claim 1 wherein said sample is analyzed to determine the presence of said illicit substance by at least one surface acoustic wave gas sensor wherein the coating is produced by technology selected from the group consisting of pulsed laser deposition, matrix assisted pulsed laser evaporation, and pulsed laser assisted surface functionalization.

4. The method of claim 1 wherein the sensor technology produces a unique electronic fingerprint to characterize the illicit substance such that the presence and concentration of the illicit substance is determined.

5. The method of claim 1 wherein said sample is analyzed to confirm the presence of said illicit substance by a spectrophotometer.

6. The method of claim 1 wherein said sample is analyzed to confirm the presence of said illicit substance by a mass spectrometer.

7. The method of claim 1 further comprising the step of recording data resulting from analysis of said sample.

8. The method of claim 1 further comprising the step of communicating data resulting from analysis of said sample to a remote system.

9. The method of claim 1 further comprising the step of analyzing data resulting from analysis of said sample with a neural classifier.

10. The method of claim 1 wherein the analysis of said sample includes comparing the results sensed in said sample against a predetermined signature library of interferents.

11. The method of claim 1 wherein the analysis of said sample includes comparing the results sensed in said sample with a predetermined signature profile of a class of illicit substances.

12. The method of claim 1 wherein the analysis of said sample includes comparing the results sensed in said sample with a predetermined signature profile of a specific illicit substance.

13. The method of claim 12 wherein the predetermined signature profile of said specific illicit substance is associated with gamma hydroxy butyrate.

14. The method of claim 1 wherein said sample is obtained by capturing exhaled breath in a vessel prior to analysis.

15. The method of claim 1 further comprising the step of dehumidifying said sample prior to analysis.

16. The method of claim 1 wherein said analysis further includes detecting exhalation with a sensor.

17. The method of claim 16 wherein said sensor is a pressure sensor.

18. The method of claim 1 wherein analysis further includes restricting the flow of exhaled breath with an air flow restrictor.

19. A method of determining the presence of an illicit substance in a subject, comprising:
obtaining a sample of exhaled breath from said subject who has possibly ingested an illicit substance;
subsequently analyzing said breath sample using gas sensor technology;
comparing the results of the analysis against a library of known illicit substances and interferents; and
identifying and confirming the presence or absence of an illicit substance in said subject.

20. A method of determining subject compliance, comprising:
obtaining a sample of exhaled breath from said subject;
subsequently analyzing said breath sample;
comparing the results of the analysis against a library of known illicit substances and interferents;
confirming the presence or absence of any illicit substance.

21. The method of claim 20 further comprising the step of identifying a baseline spectrum from said subject's breath prior to said subject's ingestion of said illicit substance.

22. An apparatus for rapidly determining subject compliance with a treatment regimen, comprising:
(a) means for receiving exhaled breath from a subject;
(b) means for determining the presence of illicit substances in said breath; and
(c) means for reporting the results.

23. The apparatus of claim 22 further comprising a mouthpiece in communication with the means for receiving exhaled breath.

24. The apparatus of claim 22 further comprising an air flow restrictor in communication with the means for receiving exhaled breath.

25. The apparatus of claim 22 further comprising an air flow sensor in communication with the means for receiving exhaled breath.

26. The apparatus of claim 25 wherein said air flow sensor is a pressure sensor.

27. The apparatus of claim 22 wherein the means for determining the presence of illicit substances in said breath comprises a sensor having a surface exposed to said subject's breath and a material selectively absorptive of a group of chemical substances of which illicit substances are members.

28. The apparatus of claim 27 wherein said sensor comprises a gas sensor.

29. The apparatus of claim 28 wherein said gas sensor is selected from the group consisting of semiconductor gas sensor technology; conductive polymer gas sensor technology; aptamer sensor technology; amplifying fluorescent polymer (AFP) sensor technology; or surface acoustic wave gas sensor technology.

30. The apparatus of claim 28 comprising at least one surface acoustic wave gas sensor wherein the coating is produced by technology selected from the group consisting of pulsed laser deposition, matrix assisted pulsed laser evaporation, and pulsed laser assisted surface functionalization.

31. The apparatus of claim 22 comprising an analysis means, coupled to the sensor, for producing an electrical signal indicative of the presence of said illicit substance.

32. The apparatus of claim 31 further comprising a stored library of interferents for comparison.

33. The apparatus of claim 31 further comprising a stored library of classes of illicit substances for comparison.

34. The apparatus of claim 31 further comprising a stored library of specific illicit substances for comparison.

35. The apparatus of claim 34 wherein said specific illicit substance is gamma hydroxy butyrate.

36. The apparatus of claim 22, wherein the analysis means are further operative to determine the approximate concentration of said illicit substance substance.

37. The apparatus of claim 22 further comprising a neural classifier.

38. The apparatus of claim 22 further comprising a means for remotely communicating the results.

39. The apparatus of claim 22 further comprising a means for storing the results.

40. A device for detecting a target substance of an illicit nature in expired breath comprising:
a surface-acoustic wave sensor capable of detecting the presence of said target substance in expired breath, wherein said sensor responds to the target substance by a shift in the resonant frequency;
an oscillator circuit having said sensor as an active feedback element; and
a frequency counter in communication with said oscillator circuit to measure oscillation frequency which corresponds to resonant frequency of the sensor;
a processor for comparing the oscillation frequency with a previously measured oscillation frequency of the target substance and determining presence and concentration of the target substance therefrom.

41. A device for detecting a target substance of an illicit nature in expired breath comprising:
a sensor having an array of polymers capable of detecting the presence of said target substance in expired breath, wherein said sensor responds to the target substance by changing the resistance in each polymer resulting in a pattern change in the sensor array;
a processor for receiving the change in resistance, comparing the change in resistance with a previously measured change in resistance, and identifying the presence of the target substance from the pattern change and the concentration of the substance from the amplitude.

42. The device of claim 41 wherein the processor comprises a neural network for comparing the change in resistance with a previously measured change in resistance to find a best match.

43. A method of determining the rate of washout of a target substance of an illicit nature in expired breath comprising:
obtaining a sample of expired breath at a first interval;
analyzing said sample with sensor technology to determine the concentration of said substance;
obtaining at least one additional sample of expired breath at a later interval;
analyzing said additional sample with sensor technology to determine the concentration of said substance; and
comparing the concentration of the first sample with the concentration of additional samples to determine rate of washout of said target substance.

44. The method of claim 43 wherein the step of determining the concentration of said substance includes the step of using sensor technology to measure metabolites of said substance.

45. The method of claim 20 further comprising the step of identifying a baseline illicit substance spectrum.
